(19) United States
(12) Patent Application Publication
(10) Pub. No.: US 2020/0031888 A1
(43) Pub. Date: Jan. 30, 2020

(54) RECOMBINANT MATURE COMPLEMENT FACTOR I

(71) Applicant: GEMINI THERAPEUTICS, Cambridge, MA (US)

(72) Inventors: David Kavanagh, Newcastle-upon-Tyne (GB); Kevin Marchbank, Newcastle-upon-Tyne (GB)

(21) Appl. No.: 16/493,701

(22) PCT Filed: Mar. 14, 2018
(86) PCT No.: PCT/US2018/022471

§ 371 Date: Sep. 12, 2019

(30) Foreign Application Priority Data
Mar. 14, 2017 (GB) 1704071.8

(51) Int. Cl. C07K 14/47; C12N 15/74

(52) U.S. Cl. CPC C07K 14/472; A61K 38/00; C12N 15/74

(57) ABSTRACT

The disclosure provides, in part, compositions comprising mature recombinant mature Complement Factor I (CFI) protein and methods of making and using those compositions.

### CROSS-REFERENCE TO RELATED APPLICATION

This application claims the benefit of priority from Great Britain Patent Application No. 1704071.8, filed on Mar. 14, 2017. The foregoing application is incorporated herein by reference in its entirety.

### TECHNICAL FIELD

Aspects of the present invention relate to a recombinant mature Complement Factor I protein, compositions comprising such proteins and methods of manufacture and uses thereof. Also included herein are methods of treating a complement-mediated disorder comprising administering a composition comprising a recombinant mature Complement Factor I protein to a patient in need thereof.

### BACKGROUND TO THE INVENTION

The complement system is a part of the innate immune system which is made up of a large number of discrete plasma proteins that react with one another to opsonize pathogens and induce a series of inflammatory responses that help to fight infection. A number of complement proteins are proteases that are themselves activated by proteolytic cleavage. There are three ways in which the complement system protects against infection. First, it generates large numbers of activated complement proteins that bind covalently to pathogens, opsonizing them for engulfment by phagocytes bearing receptors for complement. Second, the small fragments of some complement proteins act as chemo-attractants to recruit more phagocytes to the site of complement activation, and also to activate these phagocytes. Third, the terminal complement components damage certain bacteria by creating pores in the bacterial membrane.

Complement Factor I, also known as C3b/C4b inhibitor, is a serine proteinase that is essential for regulating the complement cascade. It is expressed in numerous tissues but principally by liver hepatocytes. The encoded preproprotein is cleaved to produce both heavy and light chains, which are linked by disulfide bonds to form a heterodimeric glycoprotein. This heterodimer can cleave and inactivate the complement components C4b and C3b, and it prevents the assembly of the C3 and C5 convertase enzymes. Defects in this gene cause complement factor I deficiency, an autosomal recessive disease associated with a susceptibility to pyogenic infections.

Mutations in this gene have been associated with a predisposition to atypical hemolytic uremic syndrome, a disease characterized by acute renal failure, microangiopathic hemolytic anemia and thrombocytopenia. Recently low levels of circulating CFI have been identified in individuals with very rare CFI variant genes and these individuals associated with advanced Age-Related Macular Degeneration (AMD) supporting the role of CFI in risk of AMD (Kavanagh et al (2015). AMD is the most common cause of vision loss in those aged over 50 and currently there are few treatment options. This research suggests that enhancing CFI activity in these individuals may have some therapeutic benefit.

Currently, efforts to produce compositions comprising a high percentage of recombinant mature CFI have had limited success. Typically, prior art methods result in incomplete cleavage of the proform to form the mature CFI protein. Thus, the prior art typically results in compositions comprising significant amounts of uncleaved proform protein. Furthermore, previous efforts have resulted in compositions which have reduced activity as compared to plasma-derived Complement Factor I.

It is therefore an aim of certain embodiments of the present invention to at least partially mitigate the problems associated with the prior art.

It is an aim of certain embodiments of the present invention to provide a method for producing a composition which comprises a high concentration of recombinant mature Complement Factor I.

It is an aim of certain embodiments to provide a composition comprising recombinant mature Complement Factor I for use in the treatment of complement-mediated disorders.

### SUMMARY OF THE DISCLOSURE

Unless defined otherwise, all technical and scientific terms used herein have the same meaning as commonly understood by a person skilled in the art to which to this invention belongs.

Certain aspects of the present invention provide an isolated recombinant mature Complement Factor I.

The term “isolated” as used herein refers to a biological component (such as a nucleic acid molecule or protein) that has been substantially separated or purified away from other biological components in the cell of the organism in which the component naturally occurs, i.e., other chromosomal and extra chromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins and peptides.

It is considered that the present inventors have devised a method of producing an isolated recombinant mature CFI protein which is substantially isolated from other cellular components including for example a recombinant precursor CFI protein. It is considered that prior art methods of producing a recombinant CFI protein have resulted in incomplete processing of a precursor CFI protein such that a recombinant mature CFI protein has not been substantially isolated.

In a first aspect of the present invention, there is provided a composition comprising a recombinant mature Complement Factor I (CFI) protein, wherein the recombinant mature CFI protein comprised in the composition represents greater than about 50% by weight of a total CFI protein content of the composition.

Thus, certain embodiments of the present invention relate to a recombinant mature Complement Factor I (CFI), compositions comprising recombinant mature Complement Factor I and methods of obtaining such a protein.

As used herein, the term “protein” can be used interchangeably with “peptide” or “polypeptide”, and means at least two covalently attached alpha amino acid residues linked by a peptidyl bond. The term protein encompasses purified natural products, or chemical products, which may be produced partially or wholly using recombinant or synthetic techniques. The term protein may refer to a complex of more than one polypeptide, such as a dimer or other multimer, a fusion protein, a protein variant, or derivative thereof. The term also includes modified proteins, for example, a protein modified by glycosylation, acetylation, phosphorylation, pegylation, ubiquitination, and so forth. A protein may comprise amino acids not encoded by a nucleic acid codon.

Complement Factor I is an important complement regulator. It is expressed in numerous tissues but principally by liver hepatocytes. CFI is a heterodimer in which the two chains are linked together by disulphide bond. The heavy chain contains the Factor I module, a CD5 domain and two low density lipoprotein receptor domains (LDLr). The light chain comprises a serine protease domain, the active site of which consists of a triad of His380, Asp439 and Ser525. A CFI heavy chain amino acid sequence is shown in SEQ ID. No. 1 and a CFI light chain amino acid sequence is shown in SEQ ID. No. 2 (FIG. 2).

When CFI is synthesised, it is initially made as a single chain precursor (precursor CFI protein), in which a four residue linker peptide (RRKR) connects the heavy chain to the light chain. Thus, as used herein, the term “precursor CFI protein” is used to refer to a single chain precursor Complement Factor I protein which comprises a four residue linker peptide (RRKR). Aptly, the precursor CFI protein is substantially inactive and has essentially no C3 C3b-inactivating or iC3b-degradation activity. In certain embodiments, the recombinant precursor CFI protein comprises an amino acid sequence as set forth in SEQ. ID. No. 3 (FIG. 2).

During processing, the precursor CFI protein is cleaved by a calcium-dependent serine endoprotease, furin, leaving the heavy chain and light chain of full length mature FI held together by a single disulphide bond. This protein is referred to herein as a mature CFI protein.

Thus, as used herein, the term “mature CFI protein” refers to a CFI protein which is or has been cleaved at or adjacent to a RRKR linker sequence e.g. by furin. In certain embodiments, the mature CFI protein lacks an RRKR linker sequence as compared to a precursor CFI protein, wherein the precursor CFI protein comprises a RRKR linker sequence at positions 318 to 321. In other embodiments, the mature CFI protein is cleaved adjacent to the RRKR linker sequence and therefore the mature CFI protein may comprise a light chain and a heavy chain, one or both of which comprises one or more amino acid residues of the linker sequence. In certain embodiments, the recombinant precursor CFI protein is a non-human mammalian CFI protein.

In certain embodiments, a mature CFI protein comprises a disulphide bond and wherein the recombinant mature CFI protein is cleavable into a heavy chain and a light chain upon reduction of the disulphide bond. In certain embodiments, the mature CFI protein comprises a heavy chain comprising a Factor I module, a CD5 module, an LDLr module, LDLr module and a light chain comprising a serine protease domain. In certain embodiments, the mature CFI protein is glycosylated.

As used herein, the term “recombinant precursor CFI protein” is used to refer to a precursor CFI protein as described above which is obtained using recombinant methods.

As used herein, the term “total CFI protein content” refers to a total content of the combination of recombinant mature CFI protein and a recombinant precursor CFI protein present in a single composition.

Aptly, a “recombinant mature CFI protein” is a mature CFI protein defined above which is made by recombinant expression, i.e. it is not naturally occurring or derived from plasma. Aptly, a wild-type mature CFI protein comprises two chains, each chain undergoing glycosylation which results in a total of six N-linked glycosylation sites which adds up to 3 kDa of carbohydrate to the predicted molecular weight of 85 kDa.

The recombinant mature CFI protein may have a different glycosylation pattern to a naturally-derived i.e. plasma-derived mature CFI protein.

The terms “recombinant” and “recombinant expression” are well-known in the art. The term “recombinant expression”, as used herein, relates to transcription and translation of an exogenous gene in a host organism. Exogenous DNA refers to any deoxyribonucleic acid that originates outside of the host cell. The exogenous DNA may be integrated in the genome of the host or expressed from a non-integrating element.

A recombinant protein includes any polypeptide expressed or capable of being expressed from a recombinant nucleic acid. Thus, a recombinant mature CFI protein is expressed by a recombinant DNA sequence. Aptly, the recombinant mature CFI protein has undergone post-expression processing to be cleaved at or adjacent to a RRKR linker sequence to leave a heterodimer as described herein.

In certain embodiments, the recombinant mature CFI protein represents greater than about 60% by weight of the total CFI protein content of the composition. In certain embodiments, the recombinant mature CFI protein represents greater than about 70% by weight of the total CFI protein content of the composition. In one embodiment, the recombinant mature CFI protein represents greater than about 80% by weight of the total CFI protein content of the composition.

In certain embodiments, the recombinant mature CFI protein represents greater than about 90% by weight of the total CFI protein content of the composition. Aptly, the recombinant mature CFI protein represents greater than about 95% by weight of the total CFI protein content of the composition.

In certain embodiment, the composition further comprises a recombinant precursor Complement Factor I protein, wherein the ratio of recombinant mature CFI:recombinant precursor CFI in the composition is from greater than 50:50 to 100:0.

In a second aspect of the present invention, there is provided a composition comprising a recombinant mature Complement Factor I (CFI) protein and optionally a recombinant precursor Complement Factor I protein, wherein the ratio of recombinant mature CFI:recombinant precursor CFI in the composition is from greater than 50:50 to 100:0.

In certain embodiments, the ratio of recombinant mature CFI:recombinant precursor CFI in the composition is from 60:40 to 100:0. In certain embodiments, the ratio of recombinant mature CFI:recombinant precursor CFI in the composition is from 70:30 to 100:0. In certain embodiments, the ratio of recombinant mature CFI:recombinant precursor CFI in the composition is from 80:20 to 100:0, for example from about 90:10 to 100:0, for example from 95:05 to 100:0.

In certain embodiments, the recombinant CFI protein is a human CFI protein. In certain embodiments, the recombinant mature CFI protein comprises a first amino acid molecule comprising an amino acid sequence as set forth in SEQ. ID. No. 1. In certain embodiments, the recombinant mature CFI protein comprises a first amino acid molecule comprising an amino acid sequence which has at least 80% sequence identity to the amino acid sequence as set forth in SEQ. ID. No. 1. Aptly, the % sequence identity is over the entire length of the amino acid sequence set forth in SEQ. ID. No. 1.

In certain embodiments, the recombinant mature CFI protein comprises a first amino acid sequence that is at least 90% identical to the amino acid sequence as set forth in SEQ ID NO: 1, e.g. at least 91%, 92%, 93% or 94%. In certain embodiments, the recombinant mature CFI protein comprises a first amino acid molecule comprising an amino acid sequence that is at least 95% identical to the amino acid sequence as set forth in SEQ ID NO: 1, e.g. 96%, 97%, 98%, 99% or 100% identical.

In certain embodiments, the recombinant mature CFI protein comprises a further amino acid molecule comprising an amino acid sequence as set forth in SEQ. ID. No. 2, wherein the first and further amino acid sequence are linked by a disulphide bond.

In certain embodiments, the recombinant mature CFI protein comprises a further amino acid molecule comprising an amino acid sequence which has at least 80% sequence identity to the amino acid sequence as set forth in SEQ. ID. No. 2 wherein the first and further amino acid sequence are linked by a disulphide bond. In certain embodiments, the recombinant mature CFI protein comprises an amino acid sequence that is at least 90% identical to the amino acid sequence as set forth in SEQ ID NO: 1, e.g. at least 91%, 92%, 93% or 94% identical.

In certain embodiments, the recombinant mature CFI protein comprises a further amino acid molecule comprising an amino acid sequence that is at least 95% identical to the amino acid sequence as set forth in SEQ ID NO: 2, e.g. at least 96%, 97%, 98%, 99% or 100% identical.

Thus, in certain embodiments, proteins having minor modifications in the sequence may be equally useful, provided they are functional. The terms “sequence identity”, “percent identity” and “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

Suitable programs to determine percent sequence identity include for example the BLAST suite of programs available from the U.S. government's National Center for Biotechnology Information BLAST web site (http://blast.ncbi.nlm.nih.gov/Blast.cgi). Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

In certain embodiments, the recombinant mature CFI protein may comprise an amino acid sequence comprising one or more mutations as compared to a reference sequence. In certain embodiments, the reference sequence is as shown in SEQ. ID. No. 1 and 2. In certain embodiments, the mutation may be an insertion, a deletion, or a substitution.

Substitutional variants of proteins are those in which at least one amino acid residue in the amino acid sequence has been removed and a different amino acid residue inserted in its place. The mature recombinant CFI protein of certain embodiments of the present invention can contain conservative or non-conservative substitutions.

The term “conservative substitution” as used herein relates to the substitution of one or more amino acid residues for amino acid residues having similar biochemical properties. Typically, conservative substitutions have little or no impact on the activity of a resulting protein. Screening of variants of the CFI proteins described herein can be used to identify which amino acid residues can tolerate an amino acid residue substitution. In one example, the relevant biological activity of a modified protein is not decreased by more than 25%, preferably not more than 20%, especially not more than 10%, compared with CFI when one or more conservative amino acid residue substitutions are effected.

In certain embodiments, the composition is essentially free of a furin protein or fragments thereof. Furin is a subtilisin-like proprotein convertase which cleaves protein in vivo at a minimal cleavage site of Arg-X-X-Arg. A human furin protein comprises an amino acid sequence as set forth in SEQ. ID. 4.

In certain embodiments, the composition is a pharmaceutical composition. The pharmaceutical composition further comprises one or more pharmaceutically acceptable excipients. Further details of pharmaceutical compositions are provided herein.

In a further aspect of the present invention, there is provided a method of preparing a composition comprising a recombinant mature Complement Factor I (CFI) protein, wherein the recombinant mature CFI protein represents greater than 50% by weight of a total CFI protein content of the composition, the method comprising:
a. contacting a recombinant precursor CFI protein with a furin protein or fragment thereof; and
b. incubating the recombinant precursor CFI protein with the furin protein or fragment thereof for a predetermined period of time, whereby the furin protein or fragment thereof cleaves the recombinant precursor CFI protein at or adjacent to a RRKR linker sequence site to form the recombinant mature Complement Factor I protein.

In certain embodiments, the recombinant precursor CFI protein is a human precursor CFI protein, the recombinant precursor CFI protein comprises an amino acid sequence as set forth in SEQ. ID. No: 3. In certain embodiments, the recombinant precursor CFI protein is as described herein.

In certain embodiments, the recombinant precursor CFI protein comprises a tag. In certain embodiments, the tag is a His-tag.

In certain embodiments, the method comprises expressing the recombinant precursor CFI protein prior to step (a). In certain embodiments, the method comprises expressing the recombinant precursor CFI protein in a eukaryotic cell.

In certain embodiments, the method comprises expressing the recombinant precursor CFI protein in a prokaryotic cell. Aptly, the prokaryotic cell is Escherichia coli.

In certain embodiments, the eukaryotic cell is selected from an insect, a plant, a yeast or a mammalian cell.

Suitable host cells for cloning or expressing the DNA encoding a CFI protein include prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus. Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast may be suitable cloning or expression hosts for CFI-encoding vectors. Saccharomyces cerevisiae, or common baker's yeast, is the most commonly used among lower eukaryotic host microorganisms although others may be useful.

In certain embodiments, the host cell is a mammalian host cell e.g. monkey kidney CV1 line transformed by SV40 (e.g. COS-7); human embryonic kidney line (e.g. 293 or 293 cells); baby hamster kidney cells (e.g. BHK); Chinese hamster ovary cells/−DHFR (CHO), mouse sertoli cells (e.g. TM4); monkey kidney cells (e.g. CV1); African green monkey kidney cells (e.g. VERO-76); human cervical carcinoma cells (e.g. HELA); canine kidney cells (e.g. MDCK); buffalo rat liver cells (e.g. BRL 3A); human lung cells (e.g. W138); human liver cells (e.g. Hep G2); mouse mammary tumor (MMT 060562); TRI cells, MRC 5 cells and FS4 cells. In certain embodiments, the mammalian cell is a CHO cell.

Host cells are transformed with the above-described expression or cloning vectors for antibody production and cultured in conventional nutrient media modified as appropriate for inducing promoters, selecting transformants, or amplifying the genes encoding the desired sequences.

In certain embodiments, the method comprises transforming the cell with a nucleic acid molecule encoding a precursor CFI protein. Aptly, the method comprises transforming the cell with a vector which encodes a precursor CFI protein as described herein.

“Nucleic acid molecule’ or ‘nucleic acid sequence”, as used herein, refers to a polymer of nucleotides in which the 3′ position of one nucleotide sugar is linked to the 5′ position of the next by a phosphodiester bridge. In a linear nucleic acid strand, one end typically has a free 5′ phosphate group, the other a free 3′ hydroxyl group. Nucleic acid sequences may be used herein to refer to oligonucleotides, or polynucleotides, and fragments or portions thereof, and to DNA or RNA of genomic or synthetic origin that may be single- or double-stranded, and represent the sense or antisense strand.

The term “vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and aptly, is a DNA plasmid.

Aptly, the vector may further comprise a promoter. The term “promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

In certain embodiments, the method comprises isolating the expressed recombinant precursor CFI protein prior to step (a). In certain embodiments, step (a) comprises adding the furin protein or fragment thereof to a solution comprising the expressed recombinant precursor CFI protein.

In certain embodiments, step (b) comprises incubating the furin protein or fragment thereof with the recombinant precursor CFI protein at a temperature of between about 25° C. to about 42° C.

In certain embodiments, step (b) comprises incubating the furin protein or fragment thereof with the recombinant precursor CFI protein at a temperature of between about 30° C. to about 42° C.

In certain embodiments, step (b) comprises incubating the furin protein or fragment thereof with the recombinant precursor CFI protein at a temperature of between about 35° C. to about 38° C.

In certain embodiments, step (b) comprises incubating the furin protein or fragment thereof with the recombinant precursor CFI protein in a solution having a pH of between about 5 and 7.

In certain embodiments, step (b) comprises incubating the furin protein or fragment thereof with the recombinant precursor CFI protein in a solution having a pH of between about 5 and 6.

In certain embodiments, the solution comprises calcium ions. In certain embodiments, the solution comprises calcium ions at a concentration of between about 1 mM to about 5 mM. In certain embodiments, the solution further comprises potassium ions.

In certain embodiments, step (b) comprises incubating the furin protein or fragment thereof with the recombinant precursor CFI protein for between about 5 hours and about 48 hours.

In certain embodiments, step (b) comprises incubating the furin protein or fragment thereof with the recombinant precursor CFI protein for between about 8 hours and about 20 hours.

In certain embodiments, the furin protein is a human furin protein or fragment thereof. In certain embodiments, the furin protein is a fragment of a mature furin protein. Aptly, the furin protein is a truncated furin protein which is terminated before the transmembrane domain. Aptly the truncated furin protein comprises at least one or more amino acid residues at a position at or between 595-791 that is involved in the catalytic activity of furin e.g. to cleave at a RRKR linker sequence.

In certain embodiments, the furin protein or fragment thereof is glycosylated. Aptly, the furin protein or fragment thereof is glycosylated at one or more amino acid residues selected from Asn387, Asn440 and Asn553.

In certain embodiments, the furin protein or fragment thereof has a molecular weight of 60 kDa or greater. Aptly, the furin protein or fragment thereof has a molecular weight of between about 65 to 85 kDa. In certain embodiments, the furin protein or fragment thereof comprises a tag e.g. a His tag.

In certain embodiments, the furin protein or fragment thereof comprises the amino acid sequence as set forth in SEQ. ID. No. 4 or a fragment thereof. In certain embodiments, the furin protein fragment comprises at least amino acid residues 108 to 715 of a protein comprising the amino acid sequence as set forth in SEQ. ID. No: 4.

In certain embodiments, the furin protein is a protein having at least 80%, e.g. at least 85%, 90%, 91%, 92%, 93%, 94%, 95%, 96%, 97%, 98%, 99% or 100% sequence identity with a protein having a sequence as depicted in SEQ. ID. No. 4. Aptly, the % sequence identity is over the entire length of the amino acid sequence set forth in SEQ. ID. No. 4. In certain embodiments, the furin protein is a protein having at least 80% at least 85%, 90%, 91%, 92%, 93%, 94%, 95%, 96%, 97%, 98%, 99% or 100% sequence identity with the sequence consisting of amino acid residues 108 to 715 of SEQ. ID. No. 4.

In certain embodiments, the furin protein or fragment thereof is expressed in a mammalian cell. Aptly, the method comprises obtaining a furin protein or fragment thereof which has been expressed in a mammalian cell.

In certain embodiments, the method further comprises isolating the recombinant mature CFI protein. In certain embodiments, the method further comprises purifying the isolated recombinant mature CFI protein. In certain embodiments, the recombinant mature CFI protein is as described herein.

In a further aspect of the present invention, there is a composition obtainable from the method described herein.

In a further aspect of the present invention, there is provided a composition according to aspects of the present invention for use in the treatment of a complement-mediated disorder. In certain embodiments, the composition is for use in the treatment of a C3 myopathy.

In certain embodiments, the composition is for use in the treatment of a complement-mediated disorder. In certain embodiments, the composition is for use in the treatment of a disorder associated with Complement Factor I deficiency. Such disorders may be characterised by severe and often recurrent infections.

In a further aspect of the present invention, there is provided a method of treating a complement-mediated disorder, the method comprising:
a) administering a therapeutically effective amount of a composition as described herein to a subject in need thereof.

In certain embodiments, the method is a method of treating a C3 myopathy.

In certain embodiments, the composition is for use in the treatment of a disorder associated with Complement Factor I deficiency. Such disorders may be characterised by severe and often recurrent infections.

In certain embodiments, the complement-mediated disorder is selected from age-related macular degeneration (AMD), Alzheimer's Disease, atypical haemolytic uraemic syndrome (aHUS), membranoproliferative glomerulonephritis Type 2 (MPGN2), atherosclerosis (in particular, accelerated atherosclerosis) and chronic cardiovascular disease.

In certain embodiments, the composition is for use in the treatment of a complement-associated eye condition, for example, age-related macular degeneration (AMD), choroidal neovascularization (CNV), uveitis, diabetic and other ischemia-related retinopathies, diabetic macular edema, pathological myopia, von Hippel-Lindau disease, histoplasmosis of the eye, Central Retinal Vein Occlusion (CRVO), corneal neovascularization, and retinal neovascularization.

In certain embodiments, the composition is for use in the treatment of age-related macular degeneration. Age-related Macular Degeneration (AMD) is the leading cause of blindness in the elderly worldwide. AMD is characterized by a progressive loss of central vision attributable to degenerative and neovascular changes in the macula, a highly specialized region of the ocular retina responsible for fine visual acuity. In certain embodiments, the group of complement-associated eye conditions includes age-related macular degeneration (AMD), including non-exudative (wet) and exudative (dry or atrophic) AMD, choroidal neovascularization (CNV), diabetic retinopathy (DR), and endophthalmitis.

AMD is age-related degeneration of the macula, which is the leading cause of irreversible visual dysfunction in individuals over the age of 60. Two types of AMD exist, non-exudative (dry) and exudative (wet) AMD. The dry, or nonexudative, form involves atrophic and hypertrophic changes in the retinal pigment epithelium (RPE) underlying the central retina (macula) as well as deposits (drusen) on the RPE. Patients with nonexudative AMD can progress to the wet, or exudative, form of AMD, in which abnormal blood vessels called choroidal neovascular membranes (CNVMs) develop under the retina, leak fluid and blood, and ultimately cause a blinding disciform scar in and under the retina. Nonexudative AMD, which is usually a precursor of exudative AMD, is more common. The presentation of nonexudative AMD varies; hard drusen, soft drusen, RPE geographic atrophy, and pigment clumping can be present. Complement components are deposited on the RPE early in AMD and are major constituents of drusen.

In certain embodiments, the composition described herein is for use to treat a subject. “Treatment” is an approach for obtaining beneficial or desired clinical results. For the purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

“Treatment” is an intervention performed with the intention of preventing the development or altering the pathology of a disorder. Accordingly, “treatment” refers to both therapeutic treatment and prophylactic or preventative measures in certain embodiments. Those in need of treatment include those already with the disorder as well as those in which the disorder is to be prevented. By treatment is meant inhibiting or reducing an increase in pathology or symptoms when compared to the absence of treatment, and is not necessarily meant to imply complete cessation of the relevant condition.

The terms “patient”, “subject” and “individual” may be used interchangeably and refer to either a humans or non-human mammal. Aptly, the subject is a human.

As used herein an “effective” amount or a “therapeutically effective amount” of a protein refers to a nontoxic but sufficient amount of the protein to provide the desired effect. The amount that is “effective” will vary from subject to subject, depending on the age and general condition of the individual, mode of administration, and the like. An appropriate “effective” amount in any individual case may be determined by one of ordinary skill in the art using routine experimentation.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the person skilled in the art.

Aptly, a pharmaceutical composition as described herein may contain one or more pharmaceutically acceptable excipients or carriers. In some embodiments, the composition is substantially pyrogen free or is pyrogen free. In some embodiments, the composition is sterile.

Various literature references are available to facilitate the selection of pharmaceutically acceptable carriers or excipients. See, e.g., Remington's Pharmaceutical Sciences and US Pharmacopeia: National Formulary, Mack Publishing Company, Easton, Pa. (1984); Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (Eds.) (1993); Pharmaceutical Dosage Forms: Parenteral Medications, Marcel Dekker, NY; Lieberman, et al. (Eds.) (1990) Pharmaceutical Dosage Forms: Tablets, Marcel Dekker, New York, N.Y.; Lieberman, et al. (Eds.) (1990) Pharmaceutical Dosage Forms: Disperse Systems, Marcel Dekker, NY; Weiner, Wang, E, Int. J. Pharm. 185: 129-188 (1999) and Wang W. Int. J.; Pharm. 203: 1-60 (2000), and Kotkoskie (2000) Excipient Toxicity and Safety, Marcel Dekker, New York, N.Y.

The term “pharmaceutically acceptable salt” refers to a salt of the CFI protein of embodiments of the invention. Salts include pharmaceutically acceptable salts such as acid addition salts and basic salts. Examples of acid addition salts include hydrochloride salts, citrate salts and acetate salts. Examples of basis salts include salts where the cation is selected from alkali metals, such as sodium and potassium, alkaline earth metals, such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl.

The term “solvate” in the context of the present disclosure refers to a complex of defined stoichiometry formed between a solute (e.g., a protein or pharmaceutically acceptable salt thereof according to the present disclosure) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

The pharmaceutical compositions for use in the treatment of a complement-mediated disorder can be in unit dosage form. In such form, the composition is divided into unit doses containing appropriate quantities of the active component. the unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. In certain embodiments, packaged forms include a label or insert with instructions for use. Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, and transdermal) administration. The formulations may conveniently be presented in unit dosage form and may be prepared by any of the methods well known in the art of pharmacy.

### In Vitro Uses

The bioactivity of recombinant CFI proteins and the compositions comprising such proteins can be measured in vitro using a suitable bioassay. Suitable bioassays are described below in detail, and include using surface plasmon resonance (SPR) to measure binding of the protein to CFH and measuring the ability of protein-bound CFH to interact with other relevant complement components (e.g. binding to C3b or C3d, or inducing decay of C3b.Bb).

In certain embodiments, the composition and/or recombinant mature CFI of embodiments of the present invention may be used in in vitro assays to analyse genetic variants of the CFI protein. In certain embodiments, in order to target therapy to those who will most likely receive benefit, the importance of functionally significant rare genetic variants of CFI would be advantageous. This is achieved through assays of recombinant mutant proteins compared to the wild-type protein. Overexpression of CFI in cell lines results in incomplete processing. As the precursor form of FI is not active, varying rates of processing in individual cell lines could decrease the validity of the results. Thus, the recombinant mature CFI protein of certain embodiments of the invention could be utilized in such assays.

It will be clear to a person skilled in the art that features described in relation to any of the embodiments described above can be applicable interchangeably between the different embodiments. The embodiments described above are examples to illustrate various features of the invention.

Throughout the description and claims of this specification, the words “comprise” and “contain” and variations of them mean “including but not limited to”, and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers or characteristics described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The reader's attention is directed to all papers and documents which are filed concurrently with or previous to this specification in connection with this application and which are open to public inspection with this specification, and the contents of all such papers and documents are incorporated herein by reference.

### BRIEF DESCRIPTION OF THE DRAWINGS

Embodiments of the invention are further described hereinafter with reference to the accompanying drawings, in which:

FIG. 1 depicts an overview of certain aspects of the complement system;

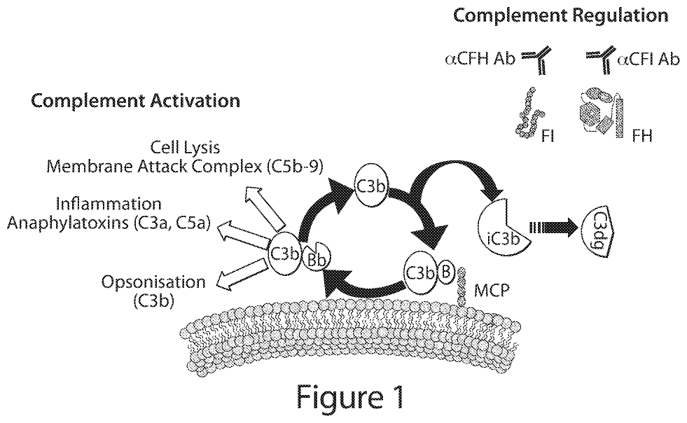

FIG. 2 depicts the amino acid sequences of proteins described herein. Particularly:
SEQ. ID. No. 1 is an amino acid sequence of human heavy chain of a mature Complement Factor I;
SEQ. ID. No. 2 is an amino acid sequence of human light chain of a mature Complement Factor I;
SEQ. ID. No. 3 is an amino acid sequence of human precursor Complement Factor I;
SEQ. ID. No. 4 is an amino acid sequence of a human furin protein; and
SEQ. ID. No. 7 is an amino acid sequence of a linker sequence of human Complement Factor I.

FIG. 3 depicts AKTA purification of WT Factor I (FI). FI was detected by measuring UV absorbance at 280 nm, as demonstrated by the blue trace. The green trace represents the imidazole gradient. The red circle highlights the point at which FI was eluted from the column. Samples of fractions corresponding to this area and surrounding fractions were run under reduced conditions on a western. There is a single band at 88 kDa and this corresponds to the proform of FI only.

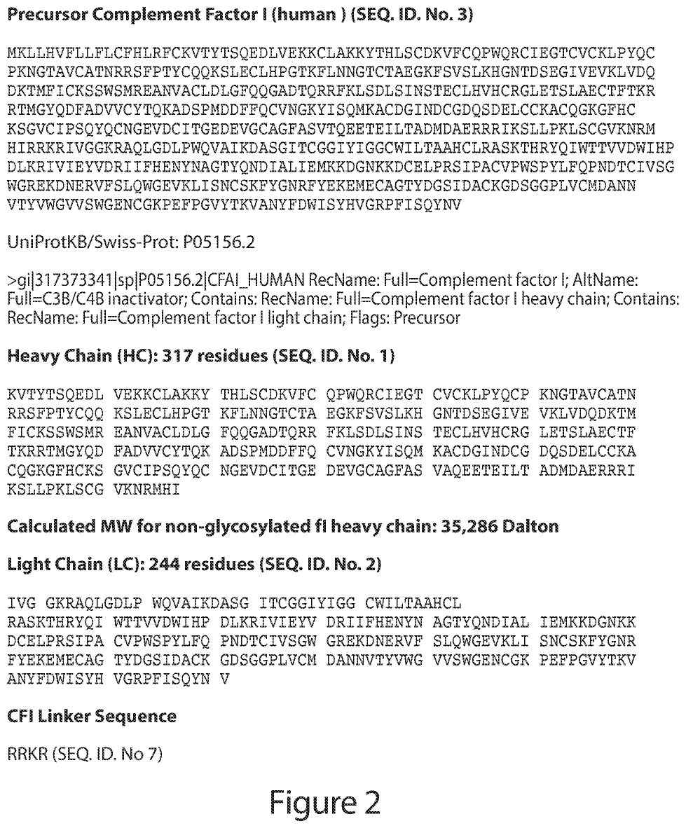

FIG. 4 is a representation of the processing of recombinant human FI in mammalian cell lines. Pro-FI undergoes processing before secretion. When CFI is expressed in cell lines, incomplete processing of the protein results in the secretion of both Pro-FI with an intact RRKR linker, and the mature FI in which the heavy and the light chain is linked only by a disulfide bond.

FIG. 5 shows appearance of FI on a Western blot. Diagram shows how different forms of FI appear on both non-reduced and reduced Western blots. Pro-FI will appear at 88 kDa under reduced and non-reduced conditions. Mature FI will appear at 88 kDa under non-reducing conditions, but when reduced will appear at 50 kDA due to breakage of the disulphide bond. FI broken down into its two constituent chains will always appear at 50 kDa under both reduced and non-reduced conditions. The light chain is not often detected on a western blot as antibodies used for detection predominantly detect heavy chain epitopes.

FIG. 6 shows Western blots to show effect of adding furin to pro-FI in sodium acetate (pH 5.0) buffer and Pro-FI in HEPES (pH 7.0) buffer. All reactions had a final concentration of 100 mM buffer and 5 mM CaCl₂. All samples were incubated for 15 hours at 37□C unless stated otherwise. Lane 1 contains purified Pro-FI before exchange into different buffers, non-incubated. Lane 2 contains Pro-FI alone in sodium acetate buffer. Lane 3 contains Pro-FI in sodium acetate buffer with furin. Lane 4 contains Pro-FI alone in HEPES buffer. Lane 3 contains Pro-FI in HEPES buffer with furin.

FIG. 7 shows a Western blot (reduced and non-reduced) to determine the minimum concentration of furin required to achieve full cleavage of Pro-FI at the RRKR linker. All reactions had a final concentration of 100 mM sodium acetate (pH 5) and 5 mM CaCl₂. Lane 1 contains Pro-FI and buffer only. Lane 2 contains Pro-FI in buffer with half of the concentration of furin in lane 1. Concentration of furin is halved a further 3 times in lanes 4, 5 and 6. Non-reduced Western confirms the nature of FI in cleavage reactions is cleaved FI.

FIG. 8 shows a Western blot (reduced) which shows the effect of changing concentration of calcium ions and potassium ions on furin efficacy. All reactions had a final concentration of 1/32 furin compared to previous experiments and 100 mM sodium acetate (pH 5) buffer. All reactions were incubated at 37□C for a period of 16 hours. The first lanes contain Pro-FI in a buffer containing 5 mM CaCl₂. The second lanes contain pro-CFI in a buffer containing 5 mM CaCl₂ with furin. The third lanes contain pro-CFI in a buffer containing 1 mM CaCl₂. The fourth lanes contain Pro-FI in a buffer containing 1 mM CaCl₂ with furin. All four lanes in the bottom western also contain 20 mM KCl. The first lanes contain Pro-FI in a buffer containing 5 mM CaCl₂. The second lanes contain pro-CFI in a buffer containing 5 mM CaCl₂ with furin. The third lanes contain pro-CFI in a buffer containing 1 mM CaCl₂. The fourth lanes contain Pro-FI in a buffer containing 1 mM CaCl₂ with furin. All four lanes in the bottom western also contain 20 mM KCl.

FIG. 9 shows the results of a C3b cofactor assay to determine the activity of Pro-FI compared to mature FI. All reactions were incubated at 37□C for 20 min. Two separate exposures are used due to low intensity of the lower bands, and too high intensity of the bands above 50 kDa. Lane 1 contains iC3b (cleaved C3b), positive control. Lane 2 contains uncleaved C3b, negative control. Lane 3 contains C3b and previously non-incubated pro-FI. Lane 4 is empty. Lane 5 contains C3b and Pro-FI. Lane 6 contains C3b and furin only, to demonstrate furin does not cleave C3b, Lane 7 contains furin alone, to demonstrate antibodies used do no cross react with furin. Lane 8 contains C3b, Pro-FI and furin (therefore cleaved FI). Appearance of α2 band in lane 1 and lane 8 only suggests cleavage of C3b took place in these lanes only. Therefore, this data suggests that only cleaved FI has activity, and Pro-FI is inactive.

FIG. 10 illustrates a western blot to determine the activity of pro-FI to mature FI. Equal samples were available for lane 4 and lane 7, which allowed a valid comparison between the activity of pro-CFI and cleaved CFI to be made. All reactions were incubated at 37□C for 20 min. Lane 1 uncleaved C3b, negative control. Lane 2 contains C3b and previously non-incubated Pro-FI. Lane 3 is empty. Lane 4 contains C3b and pro-CFI. Lane 5 contains C3b and furin only, to demonstrate furin does not cleave C3b, Lane 6 contains furin alone, to demonstrate antibodies used do no cross react with furin. Lane 7 contains C3b, Pro-FI and furin (therefore cleaved FI).

### METHODS AND MATERIALS

### Mutagenesis

The pDR2 E1F vector used for expression of recombinant pro-CFI (pro-rCFI), was provided by Dr Kevin Marchbank (Institute of Cellular Medicine Newcastle University). Site-directed mutagenesis was performed using the QuikChange site directed mutagenesis kit (Stratagene, La Jolla, Calif.) (Cat #200523) to add a 6× histidine tag to CFI cDNA in pDR2 EF1 to form pDR2 EF1α. Primers used for the mutagenesis are shown in Table 1. Full length Maxiprep sequencing was undertaken to ensure fidelity of both the wild-type and mutant vectors.

TABLE 1
Mutagenesis primers

| | |
|---|---|
| Reverse | GAGATCACAATTTTAATGATGATGATGATGATGCTTATC |
| | GTCATCGTCTACATTGTACTGAGAAATAAAAGG |
| | (SEQ. ID. NO 5) |
| Forward | CCTTTTATTTCTCAGTACAATGTAGACGATGACGATAAG |
| | CATCATCATCATCATCATTAAAATTGTGATCTC |
| | (SEQ. ID. NO 6) |

### Cell Culture

Chinese hamster ovary cells (CHO) cells were maintained in DMEM:F12 mixture (Lonza Group Ltd) supplemented with L-Glutamine (final concentration 4.5 mM, Life Technologies), penicillin and streptomycin (100 U/ml each, Life technologies) and 10% heat inactivated Fetal Bovine Serum (FBS) (Biosera). Transient transfection of CHO cells was performed using a jetPEI DNA transfection protocol.

### Cell Transfection

Cells were counted with a haemocytometer and diluted to 75,000 cells/ml. A 6 well culture plate had 2 ml of cells added per well (150,000 cells per well). 3 μg of DNA encoding the pro-CFI cDNA was diluted with sodium chloride (NaCl) to a final concentration of DNA in a volume of 100 μl. 6 μL of jetPEI reagent (Polyplus) was diluted in NaCl to a final concentration in a volume of 100 ul. The jetPEI solution was added in its entirety to the DNA solution, and this mixture was incubated for 30 minutes at room temperature. 200 μL of jetPEI/DNA mix was added per well to the cells in 1 ml of serum containing medium. Plates were then incubated at 37° C. for 24 hours. After 24 hours, the supernatant was removed from the flasks and checked for expression of CFI using a nickel pulldown assay.

### Nickel Pulldown

Hygromycin was added to incubated cells to remove non-transfected cells. Single clones were then isolated using limited dilution. Growth of cells was monitored and wells which contained a single colony of cells were established. These were transferred to separate flasks and supernatant removed to perform western blot analysis using nickel-Sepharose beads (Ni Sepharose Excel, GE Healthcare Life Sciences) to establish the best expressers of FI. 50 μL of bead slurry was placed in phosphate buffered saline (PBS) and centrifuged at 300×g to precipitate the beads, before removal of the PBS. 1 ml of cell culture supernatant was then added to the beads. The cell culture supernatant and bead mix was then incubated for 2 hours at room temperature end over end or at 4° C. overnight. After incubation the samples were centrifuged at 300×g and supernatant was removed gently so as to not disturb the pellet which should be bound to the His-tagged protein. The pellet was then washed with 20-40 mM imidazole to remove non-specifically bound proteins. After washing, samples were spun at 300×g and supernatant was removed, leaving the pellet. Pelleted nickel beads and bound protein were then subjected to western blot analysis to check for expression of pro-CFI.

The protocol followed is as follows:
1. Using 1.5 ml V bottomed tubes wash 50 ul aliquots of bead slurry (˜25 ul of beads+25 ul 20% EtOH) in PBS (each 50 ul is enough to pull down 1 ml of supernatant)
2. Spin beads at 300×g, remove PBS
3. Add 1 ml supernatant
4. Incubate for 2 hr @ RT end over end (or o/n at 4 degrees)
5. Spin at 300×g and gently remove supernatant
6. Wash with 20-40 mM Imidazole to remove non-specific binders
7. Spin at 300×g and gently remove supernatant
8. Wash with PBS
9. Spin at 300×g and gently remove supernatant leaving approx 35 ul of PBS
10. Add relevant volume of loading buffer for western ˜10 ul 5× loading buffer to account for buffer between beads
11. Boil as normal
12. Spin at 300×g remove sample and load ˜35 ul on western

### Protein Purification

Supernatant of rCFI expressing cells was collected and purified on an AKTA purifier (GE Healthcare, Piscataway, N.J.) using a 1 ml His-Trap column. A 0-0.5 M imidazole gradient in 20 mM phosphate was used to disrupt interaction of the His-tagged pro-rCFI with the His-Trap column, eluted fractions were collected. Western blots were conducted in order to determine which fractions contained pro-CFI. The fractions containing pro-rCFI were then pooled together.

### SDS Polyacrylamide Gel Electrophoresis (SDS-PAGE) and Western Blot Analysis

25 μL of sample to be studied was added to 1.5 mL tubes which contained 6.25 μL of reducing sample buffer (Thermo Scientific, 39000) or non-reducing sample buffer (Thermo Scientific, 39001). All samples were heated at 95° C. for 8 minutes before centrifugation at a speed of 13,200 rpm for 2 seconds. 10% Tris-glycine gels were made according to manufacturer's instructions (Novex, Life Sciences, EC6075BOX). Once set, gels were placed in XCell SureLock Mini-Cells (Novex, Life technologies. E10002) and the mini-cells were filled with 1× running buffer (25 mM Tris base, 192 mM Glycine, 0.1% SDS, deionised Water, pH 8.3) in both compartments. 22 μL of sample was loaded into each well of the gel. When required 14 μL of Factor I standard was loaded into a well of the gel (Comptech, A138) and used as a marker. 7 μL of MW ladder (Biolabs, P7708s) was added to at least one well of each gel. The XCell SureLock Mini-Cell was connected to a Powerpac (Bio-rad, 300V, 400 mA, 75 W) and ran for 35 minutes at 190 volts. After running, gels were transferred onto nitrocellulose membrane (Invitrogen, Life technologies, LC2001) using chilled (4° C.) 1× Tris-Glycine transfer buffer (12 mM Tris base, 96 mM Glycine, DI Water, pH 8.3, 20% Methanol). Transfer was performed by a transfer blotter run for 60 minutes at 100 volts. After transfer was complete, membranes were washed briefly with deionised water before staining with Ponceau S solution (Sigma, P7170) to determine success of transfer. Membranes were de-stained in trays placed on a rotating table. All membranes were blocked overnight at 4° C., or for 1 hour at room temperature using a solution of 5% non-fat milk powder in 1×TBST (50 mM Tris. HCl, pH 7.4, 150 mM NaCl, 0.05% Tween 20). The following antibodies were used;

For detecting pro-CFI and mature CFI: Primary antibody, sheep polyclonal Factor I (Abcam, Cambridge, Mass., ab8843) was applied at a concentration of 2.37 μg/ml for 1 hr at room temperature. Membranes were washed with Tris buffered saline tween (TBST) buffer (137 mM NaCl, 2.7 mM KCl, Tris base 19 mM, Tween) three times for 10 minutes. Secondary antibody, Rabbit polyclonal secondary antibody to sheep IgG conjugated to horse radish peroxidase (HRP) (Abcam, Cambridge, Mass.), was applied at a concentrations of 2.37 μg/ml for 1 hour at room temperature or overnight at 4° C. Blots were then washed three times for 10 min in TBST. Supersignal Chemiluminescent Substrate (Pierce, Rockford, Ill.) was applied to membranes for 1 minute before exposure to an X-ray film for varying time periods before they were developed using standard film developing techniques.

For detecting C3b and iC3b: Primary antibody, rabbit polyclonal anti-C3 antibody (Abcam) at a concentration of 1:5000 before the use of goat anti-rabbit IgG HRP antibody

### Pro-CFI Cleavage by Furin In Vitro

Experiments to optimise the in vitro cleavage of pro-rCFI by furin were carried out as detailed herein.

Purified pro-rCFI was buffer exchanged from elution buffer into 1× cleavage buffer (100 mM HEPES pH 5.2, 0.5% Triton X-100, and 1 mM CaCl₂) using a PD-10 desalting column (GE Healthcare) with a bed volume of 8.3 ml.

Furin was obtained from R & D Systems. Properties of the furin protein are provided in Table 2

TABLE 2

| | |
|---|---|
| Supplier | R & D Systems |
| Storage buffer pH | 9 |
| Presence of tag(s) | C-terminal 10 his-tag |
| Protein structure | Truncated (amino acid residues 108-715) |
| Molecular Weight | The calculated molecular weight of truncated human |
| | furin is 65 kDa. Its apparent molecular weight in |
| | SDS-PAGE gels is 65-85 kDa. |
| Source | Mouse myeloma cell line. |
| Unit definition | Measured by its ability to cleave the fluorogenic |
| | peptide substrate pER TKRAMC (Catalog # ES013). |
| | The specific activity is >125 pmol/min/μg. |

Pro-rFI was buffer exchanged from elution buffer into 1× cleavage buffer (100 mM HEPES pH 5.2, 0.5% Triton X-100, and 1 mM CaCl₂) using a PD-10 desalting column (GE Healthcare) with a bed volume of 8.3 ml.

Cleavage reactions using furin-RD were made up as detailed in Table 3 below.

TABLE 3

| | | | Cleavage | Total |
|---|---|---|---|---|
| | Furin-RD | Pro-rCFI | buffer | volume |
| Sample | (μL) | (μL) | (μL) | (μL) |
| 1A (pro-CFI only) | 0 | 234 | 141 | 375 |
| 2A (pro-CFI only | 0 | 234 | 141 | 375 |
| 3A (pro-CFI + furin) | 0 | 234 | 126 | 375 |
| 1B (pro-CFI only) | 0 | 31.2 | 18.8 | 50 |
| 2B (pro-CFI only | 0 | 31.2 | 18.8 | 50 |
| 3B (pro-CFI + furin) | 2 | 31.2 | 16.8 | 50 |
| 4B (Furin only) | 2 | 0 | 48 | 50 |

### Optimisation of Cleavage Reaction pH

In order to test the optimum pH for cleavage of pro-CFI a number of buffers with different pH values were tested. Firstly the purified pro-rCFI was exchanged from elution buffer into three buffers of differing pH using PD MidiTrap G-25 columns (GE Healthcare). Columns were equilibrated using 15 ml in total of the respective buffer which was 100 mM (sodium acetate, pH 5.0 or HEPES, pH 7 or Tris-base pH 9). 0.93 ml pro-rCFI in elution buffer was added to each column before centrifugation at 1000×g for 2 minutes. To establish whether buffer exchange was successful, 30 μL of pro-rCFI exchanged at each pH was subjected to western blot analysis as described previously. Exact quantities of reaction mixes are shown in Table 4 below.

TABLE 4
Volumes used for buffer exchange.

| | | 1M | 50 mM | | |
|---|---|---|---|---|---|
| | Pro-rCFI | corresponding | CaCl₂ | DI H₂O | Total |
| Sample | (μL) | stock buffer (μL) | (μL) | (μL) | (μL) |
| pH 5 | 30 | 2 | 5 | 13 | 50 |
| pH 7 | 30 | 2 | 5 | 13 | 50 |
| pH 9 | 30 | 2 | 5 | 13 | 50 |

Furin cleavage reactions were then set up containing 30 μL of pro-rCFI in the respective buffer and 2 μL of furin. In order to ensure the concentrations of the buffer to which the pro-rCFI had been exchanged to 2 μL of 1 M stock solution of each respective buffer was added to samples before making samples up to 50 μL with deionised water. Control reactions without furin were set up for each pH buffer. Reactions were incubated at 37□C for 15 hours. Non-incubated samples of pre-exchange, purified pro-rCFI were also set up. Quantities of each reaction are shown in Table 5.

TABLE 5
Volumes for pH optimisation of pro-rCFI cleavage by furin.

| | | | | 1M stock | | | |
|---|---|---|---|---|---|---|---|
| | | Pro-Factor I (of | | buffer (at | | | |
| | | the appropriate | Furin | the appropriate | 50 mM | H₂O | Total |
| Sample | Incubated | pH) (ul) | (ul) | pH) (ul) | CaCl₂ (ul) | (ul) | (ul) |
| Previously purified | No | 30 μL | 0 | 2 | 5 | 13 | 50 |
| Batch Pro-CFI | | (previously | | | | | |
| | | purified WT CFI) | | | | | |
| Newly purified batch | No | 30 μL | 0 | 2 | 5 | 13 | 50 |
| Pro-rCFI (before desalting) | | (newly purified | | | | | |
| | | CCFI before | | | | | |
| | | desalting) | | | | | |
| pH 5 Pro-rCFI only | Yes | 30 μL (pH 5) | 0 | 2 | 5 | 13 | 50 |
| pH 5 Pro-rCFI and furin | Yes | 30 μL (pH 5) | 2 | 2 | 5 | 11 | 50 |
| pH 7 Pro-rCFI only | Yes | 30 μL (pH 7) | 0 | 2 | 5 | 13 | 50 |
| pH 7 Pro-rCFI and furin | Yes | 30 μL (pH 7) | 2 | 2 | 5 | 11 | 50 |
| pH 9 Pro-rCFI only | No | 30 μL (pH 9) | 0 | 2 | 5 | 13 | 50 |
| pH 9 Pro-rCFI only | Yes | 30 μL (pH 9) | 0 | 2 | 5 | 13 | 50 |
| pH 9 Pro-rCFI and furin | Yes | 30 μL (pH 9) | 2 | 2 | 5 | 11 | 50 |

After incubation the samples were subjected to western blot analysis as described previously to assess the level of conversion of pro-CFI to mature CFI by detection of the constitute bands of each.

### Testing Effect of Furin Concentration on Pro-CFI Cleavage

In order to test the minimal amount of furin needed for relatively high rates of cleavage of pro-rCFI serial dilutions of furin were made up: 1:2, 1:4, 1:8 and 1:16. The diluted furin was used for cleavage reactions set up as shown in Table 6. Samples were incubated at 37° C. for 16 hours.

TABLE 6
Volumes of reactions to test furin concentration effect on cleavage rate.

| | pro-rCFI | | | | | | |
|---|---|---|---|---|---|---|---|
| | exchanged into | | Furin | 1M stock sodium | | | |
| | 100 mM sodium | Furin | dilution | acetate pH 5 | 50 mM | H₂O | Total |
| Sample | acetate pH 5 (μL) | (μL) | factor | buffer (μL) | CaCl₂ (μL) | (μL) | (μL) |
| 1 | 30 | 0 | 0 | 2 | 5 | 13 | 50 |
| 2 | 30 | 10 | 1:1 | 2 | 5 | 3 | 50 |
| 3 | 30 | 10 | 1:2 | 2 | 5 | 3 | 50 |
| 4 | 30 | 10 | 1:4 | 2 | 5 | 3 | 50 |
| 5 | 30 | 10 | 1:8 | 2 | 5 | 3 | 50 |
| 6 | 30 | 10 | 1:16 | 2 | 5 | 3 | 50 |

After incubation samples were subjected to western blot analysis as described previously.

### Testing Effect of Ion Concentration on Furin Cleavage of Pro-CFI

In order to test the effect that ion concentration had on the cleavage of pro-rCFI by furin differing potassium and calcium concentrations were tested. Furin was diluted to 1/32 of the original concentration before use in reaction mixes as detailed in Table 7 below.

TABLE 7
Ion concentration experiments, reaction volumes.

| | | | 1M | | | | | |
|---|---|---|---|---|---|---|---|---|
| | Pro- | Furin | stock sodium | | | | | |
| | rCFI pH 5 | diluted | acetate pH 5 | 10 mM | 50 mM | H₂O | KCl | Total |
| Sample | buffer (μL) | 1:16 (μL) | buffer (μL) | CaCl₂ (μL) | CaCl₂ (μL) | (μL) | 250 mM | (μL) |
| 1 | 15 | 0 | 1 | 0 | 2.5 | 6.4 | 0 | 25 |
| 2 | 15 | 2.5 | 1 | 0 | 2.5 | 4 | 0 | 25 |
| 3 | 15 | 0 | 1 | 2.5 | 0 | 6.5 | 0 | 25 |
| 4 | 15 | 2.5 | 1 | 2.5 | 0 | 4 | 0 | 25 |
| 5 | 15 | 0 | 1 | 0 | 2.5 | 4.5 | 2 | 25 |
| 6 | 15 | 2.5 | 1 | 0 | 2.5 | 2 | 2 | 25 |
| 7 | 15 | 0 | 1 | 2.5 | 0 | 4.5 | 2 | 25 |
| 8 | 15 | 2.5 | 1 | 2.5 | 0 | 2 | 2 | 25 |

### Optimised Pro-CFI Digestion Reaction Volumes

Pro-rCFI digestion was performed using the reaction mixes detailed in Table 8. Pro-rCFI was used in pH5 buffer (100 mM sodium acetate pH 5).

TABLE 8
Optimised cleavage reactions.

| | Reaction | | | | | |
|---|---|---|---|---|---|---|
| Reactant | Pro-rCFI + Furin | | Pro-rCFI only | | Furin only | |
| Pro-CFI | 15 | μL | 15 | μL | 0 | μL |
| Furin | 5 | μL | 0 | μL | 5 | μL |
| 1M pH 5 stock | 1 | μL | 1 | μL | 2.5 | μL |
| 50 mM CaCl₂ | 2.5 | μL | 2.5 | μL | 2.5 | μL |
| H₂O | 1.5 | μL | 6.5 | μL | 15 | μL |
| Total | 25 | μL | 25 | μL | 25 | μL |

### C3b Inactivation Assay: Comparison of Pro-CFI Vs Mature FI

A C3b inactivation assay was used to compare the activity of pro-rCFI and mature CFI. A sample of pro-rCFI was cleaved by furin using conditions identified in the optimisation (as detailed previously). Three control reactions were set up: 2× Pro-CFI only (incubated and non-incubated) and furin only (incubated) in order to determine whether any C3b cleavage occurred with pro-rCFI that had been cleaved by furin but subjected to the same conditions. All incubated samples were incubated at 37□ for 16 hours.

25 μl reactions were set up with a final concentration of C3b at 0.2 μg/μl. (Comptech). CFH (Comptech) was used as a cofactor and each reaction contained a final CFH concentration of 66.6 ng/μl. A positive control containing C3b and serum CFI (sFI) (Comptech) at a final concentration of 10 ng/μL (Comptech) was made up. A negative control for uncleaved C3b had no CFI and only C3b in. Two further controls of pro-rCFI only (incubated and non-incubated) were set up and also a control of furin only were set up by adding 10 μL of each respective reaction prepared previously. A further control of furin without the presence of CFH, CFI or C3b was also made up. Reactions were made up to the final volume of 25 μL using low salt buffer. The 7 reactions made up are detailed in Table 9.

TABLE 9
C3b inactivation assay reaction volumes

| | | Reactant | | | | | | | | | | |
|---|---|---|---|---|---|---|---|---|---|---|---|---|
| | | | | | | | | | | Low | | Total |
| | Sample | C3b | | sCFI | | CFH | | Pro- | | salt | | volume |
| Sample | description | [1.6 μg/μL] | | [11.11 ng/μL] | | [333 ng/μL] | | CFI | Furin | buffer | | (μL) |
| 1 | Cleaved C3b | 3 | μL | 4.5 | 5 μL | 5 | μL | — | — | 12.5 | μL | 25 μL |
| | control | | | | | | | | | | | |

| | | | | | | | | | | | |
|---|---|---|---|---|---|---|---|---|---|---|---|
| 2 | Uncleaved C3b | 3 | μL | — | 5 | μL | 10 μL | — | 7 | μL | 25 μL |
| | control | | | | | | | | | | |
| 3 | Pro-rCFI ONLY | 3 | μL | — | 5 | μL | 10 μL | — | 7 | μL | 25 μL |
| | (pre-incubated) | | | | | | | | | | |
| 4 | Cleaved rCFI | 3 | μL | — | 5 | μL | 10 μL | — | 7 | μL | 25 μL |
| | (pre-incubated) | | | | | | | | | | |
| 5 | Furin | 3 | μL | — | 5 | μL | 10 μL | 10 μL | 7 | μL | 25 μL |
| | control | | | | | | | | | | |
| 6 | Pro-rCFI ONLY | 3 | μL | — | 5 | μL | 10 μL | — | 7 | μL | 25 μL |
| | (non-incubated) | | | | | | | | | | |

| | | | | | | | | | |
|---|---|---|---|---|---|---|---|---|---|
| 7 | Furin ONLY | — | — | — | — | 10 μL | 15 | μL | 25 μL |

| | | | | | | | | |
|---|---|---|---|---|---|---|---|---|
| Final Concentration | 0.2 | μg/μL | 2 | ng/μL | 66.6 | ng/μL | — | — |

A 10 μL aliquot was removed at 20 minutes from each reaction mix. Each aliquot was added to an equal volume of 1× laemelli buffer and western blot analysis was performed as outlined previously using the antibodies detailed for detecting C3b. Activity of rCFI was determined by generation and intensity of the α1 and α2 bands upon developing of x-ray images.

Also ran on some gels was a sample of inactivated (cleaved) C3b (iC3B) to act as a marker for C3b cleavage products.

### Results & Discussion

### Factor I Purification

Supernatant of rCFI expressing cells was collected and purified as described herein. Collected fractions were run on a polyacrylamide gel under reducing conditions before western blotting of the gel. The presence of rCFI is confirmed by the band at a molecular weight of 88 kDa, corresponding to the MW of pro-rCFI (uncleaved). This is further confirmed by the absence of a band corresponding to a molecular weight of 50 kDa which would be expected from cleaved mature CFI. The concentration of the rCFI was determined by ELISA testing to be 0.6 ng/μL.

### Factor I Cleavage Optimisation

Cleavage of the ³¹⁸RRKR³²¹ cleavage site was optimised by testing a range of conditions, to ensure that the maximum level of cleavage of pro-rCFI to mature rCFI was achieved in vitro. All samples were subjected to polyacrylamide gel electrophoresis in reducing and non-reducing conditions to allow the distinction between mature rCFI and the heavy chain alone which may be dissociated due to degradation of the protein.

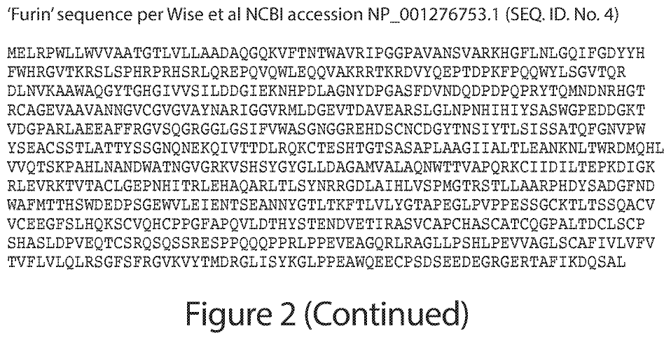

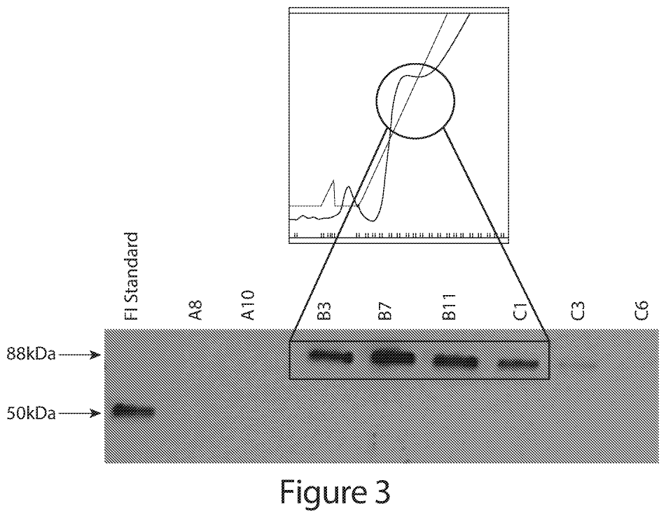

Under non-reducing conditions, both the pro-rCFI and mature rCFI should have a MW of approximately 88 kDa; when Pro-rCFI is reduced, it should remain at 88 kDa due to the existence of the RRKR linker; Mature rCFI should separate into the heavy chain (50 kDa) and light chain (37 kDa) as the di-sulphide bridge between the two chains is reduced. The light chain is not often detected on a western blot as antibodies used for detection predominantly detect heavy chain epitopes. FIG. 4 summarizes the processing of CFI in mammalian cells and demonstrates the effect of reduction on the different forms. FIG. 5 provides a diagram of how different forms of CFI are expected to appear on a western blot under both reducing and non-reducing conditions.

### Optimisation of pH for Cleavage of Pro-rCFI by Furin

After incubation with furin it can be seen from the western blots shown in FIGS. 5A (reducing conditions) and 5B (non-reducing conditions) that no pro-rCFI or mature rCFI is detectable when the reaction is performed at pH 7 (lanes 4 and 5) as is indicated by the absence of bands at ˜88 and/or 50 kDa. When the reaction was performed at pH5 the presence of a band at ˜50 kDa in FIG. 5A and absence of a band at ˜88 kDa indicates that all detectable amounts of the pro-rCFI has been cleaved to the mature CFI form.

A broad pH is provided in the prior art for cleavage of pro-CFI. These experiments show that the pH of the reaction can help maximize the cleavage of pro-rCFI to the mature form. It has been suggested that slightly acidic pH may help to increase the rate of proteolytic cleavage due to conformational change that may help to expose the cleavage site. The data here indicates that a high rate of cleavage occurs at pH 5 but other pH values may also allow for a high rate of cleavage depending on other reaction conditions and reactants that may be used.

### Optimisation of Furin Concentration

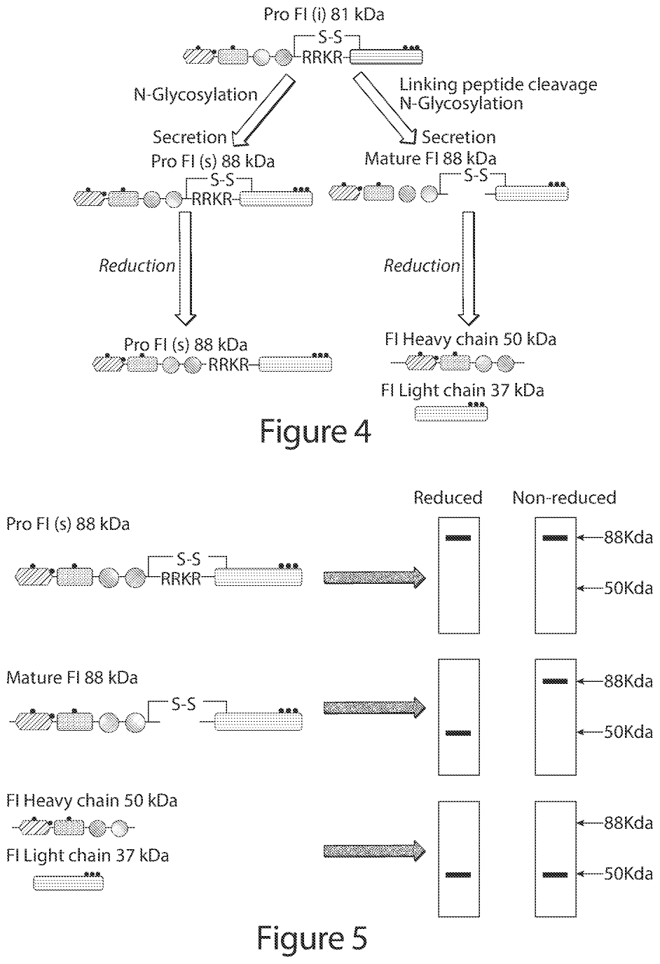

It can be seen from FIGS. 6A and 6B that even at low concentrations furin is able to provide a high rate of cleavage of pro-CFI. This can be seen by the presence of a band in the western blot performed in reducing conditions (FIG. 6A) at ˜50 kDa which corresponds to cleaved mature rCFI and the absence of a band at ˜88 kDa which corresponds to uncleaved pro-CFI. Even when furin is diluted by a factor of 16 a relatively high rate of cleavage is observed.

Cleavage by furin is confirmed by the absence of a band at ˜50 kDa in FIG. 6A lane 1 which corresponds to a pro-rCFI control. If degradation of the protein was the cause of the band seen at ˜50 kDa it would be expected to be seen for the pro-rCFI only control as well.

### Optimisation of Ion Concentration

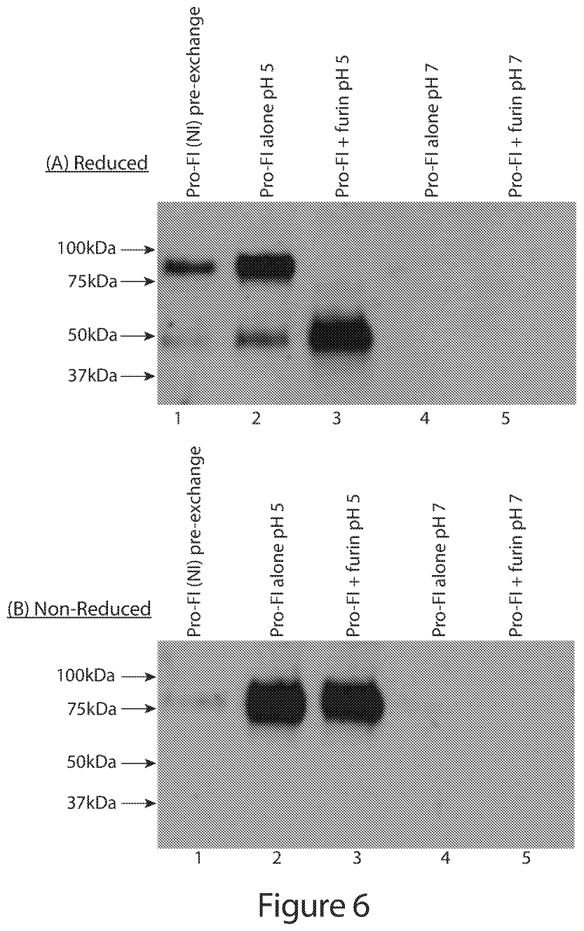

The results indicate that the regardless of potassium ion concentration, calcium ions enhances the rate of cleavage. This can be seen in FIG. 7A by the presence of a band at ˜50 kDa corresponding to cleaved mature rCFI in lane 2. The corresponding band in lane 4 which shows the products of a cleavage reaction performed with a lower (1 mM CaCl₂) calcium ion concentration and is less intense indicating that a higher (5 mM CaCl₂) calcium ion concentration may increase the cleavage rate.

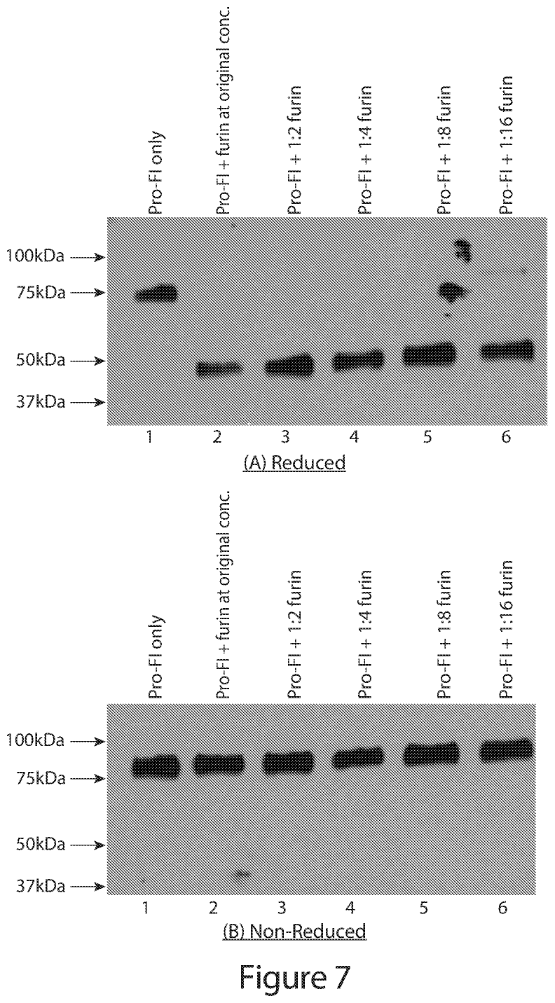

It can be seen when comparing the western blots shown in FIGS. 8A and 8B that the presence of potassium ions may help increase the rate of pro-rCFI cleavage by furin by the fact that the bands corresponding to cleaved mature rCFI (˜50 kDa) in lanes 2 and 4 of FIG. 8B have a greater intensity than that seen in FIG. 8A.

It is noted though that for shorter incubation times the presence of potassium ions may help speed up the cleavage reaction allowing for faster cleaving of all of the pro-CFI to mature CFI.

Following the optimisation tests the reaction mix and condition for cleavage of pro-CFI using furin was as given in Table 10:

TABLE 10
Optimised furin cleavage of pro-FI

| | Pro-rCFI + Furin | |
|---|---|---|
| Pro-CFI | 15 | μL |
| Furin | 5 | μL |
| 1M pH 5 stock | 1 | μL |
| 50 mM CaCl₂ | 2.5 | μL |
| H₂O | 1.5 | μL |
| Total | 25 | μL |

Pro-rCFI was first exchanged into 100 mM sodium acetate pH 5. Samples were incubated at 37° C. for 16 hours. Potassium ions were not included in the reaction as the effect they had when using the given reaction conditions was considered negligible.

### C3b Inactivation Assay: Comparison of Pro-rCFI Vs Mature CFI

In order to test then vitro activity of the in vitro cleaved mature rCFI a C3b inactivation assay was performed. It is expected that if CFI is in the active mature form it will cleave C3b into its inactive state iC3b by cleavage of the α chain to produce two chains with molecular weights of 68 kDa (α1) and 46 kDa (α2) the α2 chain is further cleaved to a final molecular weight of 43 kDa. This change in the structure of C3b can be seen by analysing the products of an C3b inactivation reaction using a western blot and comparing the intensity and occurrence of a band that corresponds to the α chain and the intensity and occurrence of bands that correspond to the α1 and α2 chains. This therefore allows for the activity of the mature CFI to cleave C3b to be accessed.

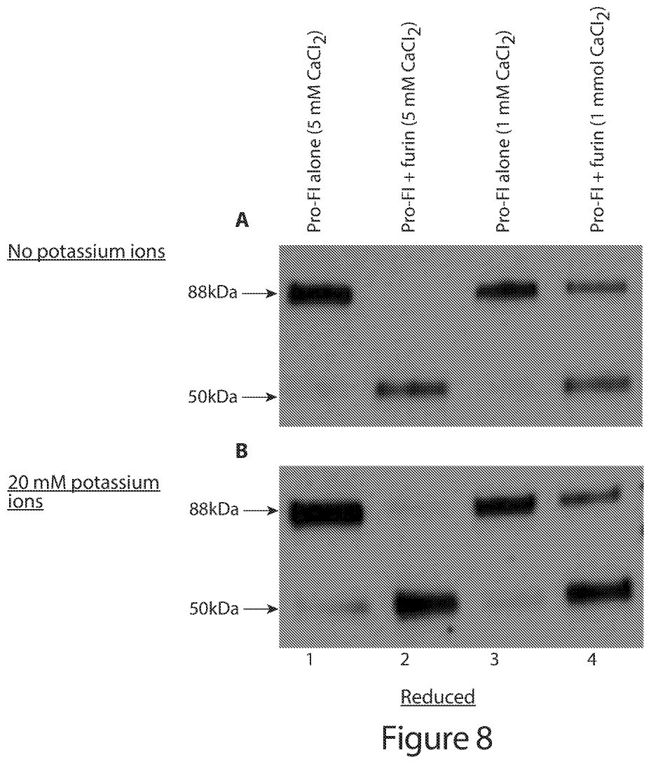

It can be seen from FIG. 9 that when compared to control experiments (lanes 2, 3, 4, 5, 6, and 7) lane 8 which corresponds to a sample containing the in vitro cleaved mature rCFI, is the only sample containing bands that correspond to the α1 and α2 chains. This scan be confirmed by comparing the western blot bands seen for the iC3b cleaved by incubation with mature sCFI.

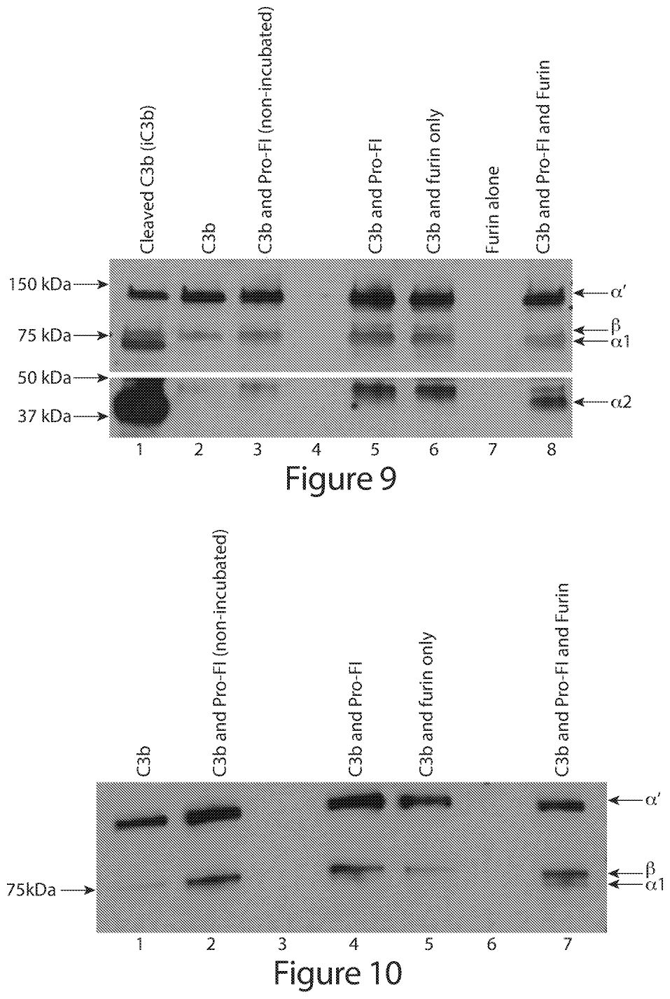

The separation of the β and α1 bands was not as defined as possible and so a second western blot was performed. FIG. 10 shows the western blot analysis with the separation of the β and α1 bands. The activity of the in vitro cleaved rCFI can be seen to be comparable to sCFI indicating that the amount of cleavage of pro-CFI to CFI is relatively high.

1. A composition comprising a recombinant mature Complement Factor I (CFI) protein, wherein the recombinant mature CFI protein comprised in the composition represents greater than about 50% by weight of a total CFI protein content of the composition.

2. The composition according to claim 1, wherein the recombinant mature CFI protein represents greater than about 60% by weight of the total CFI protein content of the composition.

3. The composition according to claim 1 or claim 2, wherein the recombinant mature CFI protein represents greater than about 70% by weight of the total CFI protein content of the composition.

4. The composition according to any preceding claim, wherein the recombinant mature CFI protein represents greater than about 80% by weight of the total CFI protein content of the composition.

5. The composition according to any preceding claim, wherein the recombinant mature CFI protein represents greater than about 90% by weight of the total CFI protein content of the composition.

6. The composition according to any preceding claim, wherein the recombinant mature CFI protein represents greater than about 95% by weight of the total CFI protein content of the composition.

7. The composition according to any preceding claim, which optionally further comprises a recombinant precursor Complement Factor I protein, wherein the ratio of recombinant mature CFI:recombinant precursor CFI in the composition is from greater than 50:50 to 100:0.

8. A composition comprising a recombinant mature Complement Factor I (CFI) protein and optionally a recombinant precursor Complement Factor I protein, wherein the ratio of recombinant mature CFI:recombinant precursor CFI in the composition is from greater than 50:50 to 100:0.

9. The composition according to claim 7 or claim 8, wherein the ratio of recombinant mature CFI:recombinant precursor CFI in the composition is from 60:40 to 100:0.

10. The composition according to any of claims 7 to 9, wherein the ratio of recombinant mature CFI:recombinant precursor CFI in the composition is from 70:30 to 100:0.

11. The composition according to any of claims 7 to 10, wherein the ratio of recombinant mature CFI:recombinant precursor CFI in the composition is from 80:20 to 100:0.

12. The composition according to any of claims 7 to 11, wherein the ratio of recombinant mature CFI:recombinant precursor CFI in the composition is from 90:10 to 100:0.

13. The composition according to any of claims 7 to 12, wherein the ratio of recombinant mature CFI:recombinant precursor CFI in the composition is from 95:05 to 100:0.

14. The composition according to any preceding claim, wherein the recombinant CFI protein is a human CFI protein.

15. The composition according to any preceding claim, wherein the recombinant mature CFI protein comprises a first amino acid molecule comprising an amino acid sequence as set forth in SEQ. ID. No. 1.

16. The composition according to any of claims 1 to 14, wherein the recombinant mature CFI protein comprises a first amino acid molecule comprising an amino acid sequence which has at least 80% sequence identity to the amino acid sequence as set forth in SEQ. ID. No. 1.

17. The composition according to claim 16, wherein the recombinant mature CFI protein comprises a first amino acid sequence that is at least 90% identical to the amino acid sequence as set forth in SEQ ID NO: 1.

18. The composition according to claim 17, wherein the recombinant mature CFI protein comprises a first amino acid molecule comprising an amino acid sequence that is at least 95% identical to the amino acid sequence as set forth in SEQ ID NO: 1.

19. The composition according to any preceding claim, wherein the recombinant mature CFI protein comprises a further amino acid molecule comprising an amino acid sequence as set forth in SEQ. ID. No. 2, wherein the first and further amino acid sequence are linked by a disulphide bond.

20. The composition according to any of claims 1 to 18, wherein the recombinant mature CFI protein comprises a further amino acid molecule comprising an amino acid sequence which has at least 80% sequence identity to the amino acid sequence as set forth in SEQ. ID. No. 2 wherein the first and further amino acid sequence are linked by a disulphide bond.

21. The composition according to claim 20, wherein the recombinant mature CFI protein comprises an amino acid sequence that is at least 90% identical to the amino acid sequence as set forth in SEQ ID NO: 1.

22. The composition according to claim 21, wherein the recombinant mature CFI protein comprises a further amino acid molecule comprising an amino acid sequence that is at least 95% identical to the amino acid sequence as set forth in SEQ ID NO: 2.

23. The composition according to any preceding claim, which is essentially free of a furin protein.

24. The composition according to any preceding claim, which is a pharmaceutical composition.

25. The composition according to claim 20, which further comprises one or more pharmaceutically acceptable excipients.

26. The composition according to any preceding claim for use in the treatment of a complement-mediated disorder.

27. The composition according to claim 22 for use in the treatment of a C3 myopathy.

28. The composition according to claim 22 for use in the treatment of a complement-mediated disorder, wherein the complement-mediated disorder is selected from age-related macular degeneration, Alzheimer's Disease, atypical haemolytic uraemic syndrome, membranoproliferative glomerulonephritis Type 2 (MPGN2), atherosclerosis (in particular, accelerated atherosclerosis) and chronic cardiovascular disease.

29. A method of preparing a composition comprising a recombinant mature Complement Factor I (CFI) protein, wherein the recombinant mature CFI protein represents greater than 50% by weight of a total CFI protein content of the composition, the method comprising:
a. contacting a recombinant precursor CFI protein with a furin protein or fragment thereof; and
b. incubating the recombinant precursor CFI protein with the furin protein or fragment thereof for a predetermined period of time, whereby the furin protein or fragment thereof cleaves the recombinant precursor CFI protein at or adjacent to a RRKR linker sequence site to form the recombinant mature Complement Factor I protein.

30. The method according to claim 29, wherein the recombinant precursor CFI protein is a human precursor CFI protein.

31. The method according to claim 29 or claim 30, wherein the recombinant precursor CFI protein comprises a tag.

32. The method of claim 31, wherein the tag is a His-tag.

33. The method according to any of claims 29 to 32, which further comprises expressing the recombinant precursor CFI protein prior to step (a).

34. The method according to claim 33, which comprises expressing the recombinant precursor CFI protein in a eukaryotic cell.

35. The method according to claim 33, which comprises expressing the recombinant precursor CFI protein in a prokaryotic cell.

36. The method of claim 35, wherein the prokaryotic cell is Escherichia coli.

37. The method according to claim 34, wherein the eukaryotic cell is selected from an insect, a yeast or a mammalian cell.

38. The method according to claim 37, wherein the mammalian cell is a CHO cell.

39. The method according to any of claims 29 to 38, which comprises isolating the expressed recombinant precursor CFI protein prior to step (a).

40. The method according to any of claims 29 to 39, wherein step (a) comprises adding the furin protein or fragment thereof to a solution comprising the expressed recombinant precursor CFI protein.

41. The method according to any of claims 29 to 40, wherein step (b) comprises incubating the furin protein or fragment thereof with the recombinant precursor CFI protein at a temperature of between about 25° C. to about 42° C.

42. The method according to any of claims 29 to 41, wherein step (b) comprises incubating the furin protein or fragment thereof with the recombinant precursor CFI protein at a temperature of between about 30° C. to about 42° C.

43. The method according to any of claims 29 to 41, wherein step (b) comprises incubating the furin protein or fragment thereof with the recombinant precursor CFI protein at a temperature of between about 35° C. to about 38° C.

44. The method according to any of claims 29 to 43, wherein step (b) comprises incubating the furin protein or fragment thereof with the recombinant precursor CFI protein in a solution having a pH of between about 5 and 7.

45. The method according to claim 44, wherein step (b) comprises incubating the furin protein or fragment thereof with the recombinant precursor CFI protein in a solution having a pH of between about 5 and 6.

46. The method according to claim 44 or claim 45, wherein the solution comprises calcium ions.

47. The method according to claim 46, wherein the solution comprises calcium ions at a concentration of between about 1 mM to about 5 mM.

48. The method according to any of claims 44 to 47, wherein the solution further comprises potassium ions.

49. The method according to any of claims 29 to 48, wherein step (b) comprises incubating the furin protein or fragment thereof with the recombinant precursor CFI protein for between about 5 hours and about 48 hours.

50. The method according to any of claims 29 to 49, wherein step (b) comprises incubating the furin protein or fragment thereof with the recombinant precursor CFI protein for between about 8 hours and about 20 hours.

51. The method according to any of claims 29 to 50, wherein the furin protein or fragment thereof comprises the amino acid sequence as set forth in SEQ. ID. No. 4 or a fragment thereof.

52. The method according to claim 51, wherein the furin protein fragment comprises at least amino acid residues 108 to 715 of a protein comprising the amino acid sequence as set forth in SEQ. ID. No: 4.

53. The method according to any of claims 29 to 52, which further comprises isolating the recombinant mature CFI protein.

54. The method according to claim 53, which further comprises purifying the isolated recombinant mature CFI protein.

55. The method according to any of claims 29 to 53, wherein the recombinant precursor CFI protein comprises an amino acid sequence as set forth in SEQ. ID. No: 3.

56. The method according to any of claims 29 to 54, wherein the recombinant precursor CFI protein comprises an amino acid sequence as set forth in SEQ. ID. No. 3.

57. A composition obtainable from the method of any of claims 29 to 56.

58. A method of treating a complement-mediated disorder, the method comprising:
a. administering a therapeutically effective amount of a composition according to any of claims 1 to 28 or claim 57 to a subject in need thereof.

59. The method according to claim 58, which is a method of treating a C3 myopathy.

60. The method according to claim 59, which is a method of treating a complement-mediated disorder, wherein the complement-mediated disorder is selected from age-related macular degeneration, Alzheimer's Disease, atypical haemolytic uraemic syndrome, membranoproliferative glomerulonephritis Type 2 (MPGN2), atherosclerosis (in particular, accelerated atherosclerosis) and chronic cardiovascular disease.

61. The method according to claim 60, which is a method of treating age-related macular degeneration.

62. A pharmaceutical composition comprising the composition of any one of claims 1-28, and a pharmaceutically acceptable carrier.

63. The pharmaceutical composition of claim 62, wherein the composition is substantially pyrogen free.

64. The pharmaceutical composition of claim 62 or 63, wherein the composition is sterile.


SEQUENCE LISTING (7 sequences)

SEQ ID NO: 1 - protein, 317 aa, Homo Sapiens
KVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVAQEETEILTADMDAERRRIKSLLPKLSCGVKNRMHI

SEQ ID NO: 2 - protein, 244 aa, Homo Sapiens
IVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 3 - protein, 583 aa, Homo Sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 4 - protein, 794 aa, Homo Sapiens
MELRPWLLWVVAATGTLVLLAADAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQACVVCEEGFSLHQKSCVQHCPPGFAPQVLDTHYSTENDVETIRASVCAPCHASCATCQGPALTDCLSCPSHASLDPVEQTCSRQSQSSRESPPQQQPPRLPPEVEAGQRLRAGLLPSHLPEVVAGLSCAFIVLVFVTVFLVLQLRSGFSFRGVKVYTMDRGLISYKGLPPEAWQEECPSDSEEDEGRGERTAFIKDQSAL

SEQ ID NO: 5 - DNA, 72 bp, Artificial
  Primer
gagatcacaattttaatgatgatgatgatgatgcttatcgtcatcgtctacattgtactgagaaataaaagg

SEQ ID NO: 6 - DNA, 72 bp, Artificial
  Primer
ccttttatttctcagtacaatgtagacgatgacgataagcatcatcatcatcatcattaaaattgtgatctc

SEQ ID NO: 7 - protein, 4 aa, Artificial
  Linker Sequence
RRKR